(19) United States
(12) Patent Application Publication
(10) Pub. No.: US 2018/0360427 A1
(43) Pub. Date: Dec. 20, 2018

(54) ULTRASONIC DIAGNOSTIC APPARATUS AND MEDICAL IMAGE PROCESSING APPARATUS

(71) Applicant: Canon Medical Systems Corporation, Otawara-shi (JP)

(72) Inventors: Fumiki NAKANO, Utsunomiya (JP); Yasuko FUJISAWA, Nasushiobara (JP)

(73) Assignee: Canon Medical Systems Corporation, Otawara-shi (JP)

(21) Appl. No.: 16/007,296

(22) Filed: Jun. 13, 2018

(30) Foreign Application Priority Data
Jun. 14, 2017 (JP) 2017-116962

(51) Int. Cl. A61B 8/08; A61B 8/00; G06T 7/00; G06T 7/73

(52) U.S. Cl. CPC A61B 8/5253; A61B 8/4263; A61B 8/463; A61B 8/5223; A61B 8/466; G06T 2207/30204; A61B 8/468; G06T 7/0014; G06T 7/74; G06T 2207/10136; G06T 2207/30096; A61B 8/5261

(57) ABSTRACT

According to one embodiment, a ultrasound diagnostic apparatuses includes processing circuitry. The processing circuitry configured to obtain an ultrasonic image assigned with incidental information as imaging information, the incidental information including at least one of a body mark and a probe mark, calculate position specifying information for specifying an imaging position with respect to the ultrasonic image by performing image recognition of the incidental information assigned to the ultrasonic image, extract from a plurality of first images obtained in a past, a second image assigned with incidental information including position specifying information corresponding to the calculated position specifying information and display the second image.

### CROSS-REFERENCE TO RELATED APPLICATIONS

This application is based upon and claims the benefit of priority from the prior Japanese Patent Application No. 2017-116962, filed Jun. 14, 2017, the entire contents of which are incorporated herein by reference.

### FIELD

Embodiments described herein relate generally to an ultrasonic diagnostic apparatus and a medical image processing apparatus.

### BACKGROUND

In the observation of ultrasonic images, there is a need of reading a similar image that was imaged in the past from the same position and comparing an ultrasonic image under observation with the read similar image.

As one of examples of a method of reading a similar image, the first method compares pixel value distributions based on pixel values of ultrasonic images and extracts a past image with a similar content. However, even if the same region in the body is imaged, a pixel value distribution of an ultrasonic image as a comparison target may depend on how a probe is applied or what imaging parameter is used. Thus, the detection accuracy is not absolute. Furthermore, since the calculation for a comparison of image feature amount based on a pixel value requires a large load, the process for extracting a similar image based on such a comparison requires a lot of time when the amount of past images becomes enormous.

The second method, for example, refers to incidental information such as a DICOM (Digital Imaging and Communications in Medicine) tag assigned to each ultrasonic image, and extracts a past image having similar incidental information. According to this second method, a similar image is extracted by making a comparison of an examination region or body mark ID assigned to each body mark. However, how to assign body mark IDs differs for each manufacturer or product version. Thus, the second method cannot handle the case where any of past images as comparison targets include a body mark ID of a different manufacture or a different product version. Furthermore, even when the same region is examined, if an actual imaging position differs each time, the same region is not always imaged when viewed as an ultrasonic image.

Thus, there is a problem in terms of the search accuracy and the search efficiency in both the method according to a comparison of image feature amount based on a pixel value and the method according to a comparison of incidental information.

### BRIEF DESCRIPTION OF THE DRAWING(S)

FIG. 1 is a view showing the structure of an ultrasonic diagnostic apparatus according to the first embodiment.

FIG. 2 is a flowchart showing the operation of a control circuitry at the time when the ultrasonic diagnostic apparatus according to the first embodiment associates a sample model with an ultrasonic image data piece.

FIG. 3 is a view showing an ultrasonic image that includes a body mark region and is displayed on a display according to the first embodiment.

FIG. 4 is a view illustrating sample models registered in sample-model information according to the first embodiment.

FIG. 5 is a view illustrating specifically how the ultrasonic diagnostic apparatus according to the first embodiment associates sample models with a human body model.

FIG. 6 is a view illustrating specifically how the ultrasonic diagnostic apparatus according to the first embodiment calculates imaging information based on a shape of probe mark included in a body mark of a sample model.

FIG. 7 is a view illustrating specifically how the ultrasonic diagnostic apparatus according to the first embodiment associates a probe mark with a human body model.

FIG. 8 is a flowchart showing the operation of a control circuitry at the time when the ultrasonic diagnostic apparatus according to the first embodiment searches for a past image similar to an ultrasonic image of an obtained ultrasonic image data piece.

FIG. 9 is a view illustrating specifically how the ultrasonic diagnostic apparatus according to the first embodiment correlates a probe mark with a sample model and a human body model.

FIG. 10 is a view showing the structure of an ultrasonic diagnostic apparatus according to the second embodiment.

FIG. 11 is a flowchart showing the operation of a control circuitry at the time when the ultrasonic diagnostic apparatus according to the second embodiment searches for a past image having a sample probe mark similar to a designated sample probe mark.

FIG. 12 is a view showing a human body model displayed on a display according to the second embodiment.

FIG. 13 is a view showing the structure of an ultrasonic diagnostic apparatus according to the third embodiment.

FIG. 14 is a flowchart showing the operation of a control circuitry at the time when the ultrasonic diagnostic apparatus according to the third embodiment searches for a past image similar to an ultrasonic image of an obtained ultrasonic image data piece.

### DETAILED DESCRIPTION

According to one embodiment, a ultrasound diagnostic apparatuses includes processing circuitry. The processing circuitry configured to obtain an ultrasonic image assigned with incidental information as imaging information, the incidental information including at least one of a body mark and a probe mark, the body mark corresponding to an imaged region at a time when a subject is imaged with an ultrasonic probe, the probe mark defining a position where the ultrasonic probe locates on the body mark, calculate position specifying information for specifying an imaging position with respect to the ultrasonic image by performing image recognition of the incidental information assigned to the ultrasonic image, extract from a plurality of first images obtained in a past, a second image assigned with incidental information including position specifying information corresponding to the calculated position specifying information and display the second image.

(Translation is omitted.)

Embodiments will be described below with reference to the drawings.

### First Embodiment

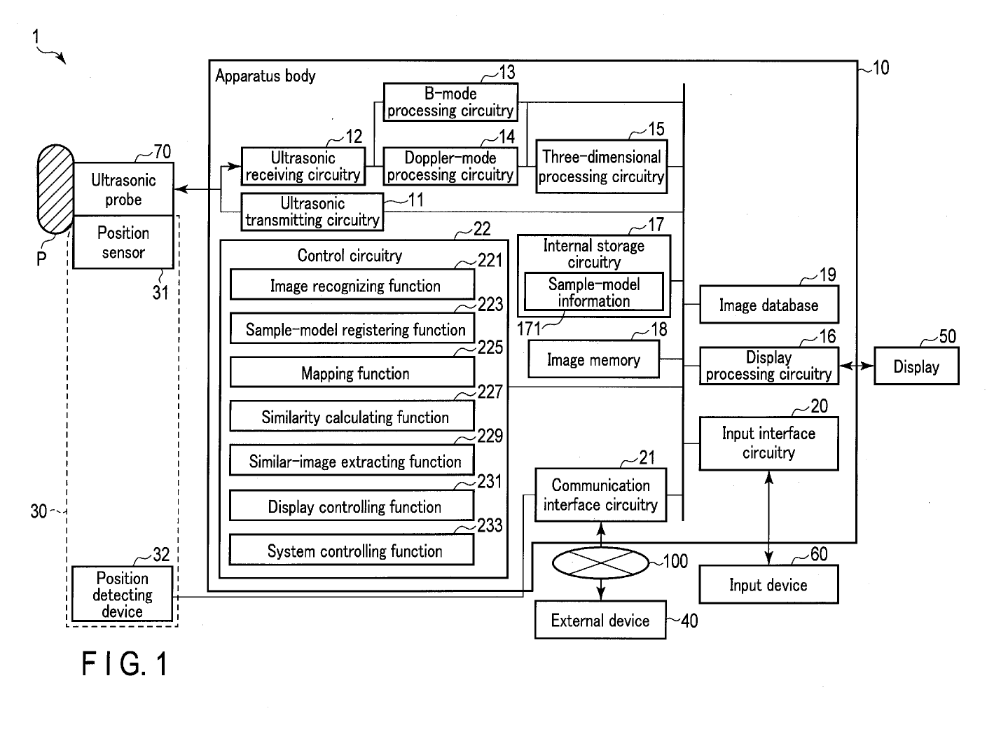

An ultrasonic diagnostic apparatus 1 according to the first embodiment will be described with reference to the block diagram shown in FIG. 1.

As shown in FIG. 1, the ultrasonic diagnostic apparatus 1 includes an apparatus body 10, an ultrasonic probe 70, a position sensor system 30, a display 50, and an input device 60. The apparatus body 10 is connected to an external device 40 via a network 100. The apparatus body 10 is also connected to the position sensor system 30, the display 50, and the input device 60.

The position sensor system 30 is a system for obtaining three-dimensional position information of the ultrasonic probe 70 and an ultrasonic image. The position sensor system 30 includes a position sensor 31 and a position detecting device 32.

The position sensor system 30 obtains three-dimensional position information of the ultrasonic probe 70 by attaching the position sensor 31 to the ultrasonic probe 70. Examples of the position sensor 31 to be attached include a magnetic sensor, an infrared sensor, a target for an infrared camera, etc. Alternatively, the position sensor system 30 may obtain three-dimensional position information of the ultrasonic probe 70 via a gyro sensor (angular speed sensor) by incorporating it into the ultrasonic probe 70.

Furthermore, the position sensor system 30 may obtain three-dimensional position information of the ultrasonic probe 70 by imaging the ultrasonic probe 70 with a camera and performing an image recognition process to the imaged image. The position sensor system 30 may obtain position information of the ultrasonic probe 70 by holding it with a robot arm and obtaining the three-dimensional position of this robot arm as such position information of the ultrasonic probe 70.

Hereinafter, described as an example is the case where the position sensor system 30 obtains position information of the ultrasonic probe 70 using the magnetic sensor.

Specifically, the position sensor system 30 further includes a magnetic generator (not shown) having, e.g., a magnetic generating coil. The magnetic generator forms a magnetic field toward the outside with the magnetic generator itself as a center. In the formed magnetic field, a magnetic space which provides secure position accuracy is defined. Accordingly, the magnetic generator may be arranged in a manner so that a living body as an examination target is included in the magnetic space in which the position accuracy is secure. The position sensor 31 assigned to the ultrasonic probe 70 detects the strength and inclination of a three-dimensional magnetic field formed by the magnetic generator. This enables the position sensor system 30 to obtain the position and direction of the ultrasonic probe 70. The position sensor 31 outputs the detected strength and inclination of magnetic field to the position detecting device 32.

Based on the strength and inclination of magnetic field detected by the position sensor 31, the position detecting device 32 calculates, for example, a position (position (x, y, z) and rotation angle (θx, θy, θz) of a scanning surface) of the ultrasonic probe 70 in a three-dimensional space with a predetermined position as an origin. In making this calculation, the predetermined position is set as a position where the magnetic generator, for example, is arranged. The position detecting device 32 transmits position information regarding a calculated position (x, y, z, θx, θy, θz) to the apparatus body 10.

The position information obtained as described above is correlated with the ultrasonic image data piece of ultrasonic wave that is transmitted and received by the ultrasonic probe 70, by means of time synchronization, etc. In this manner, position information can be given to the ultrasonic image data piece.

The ultrasonic probe 70 includes a plurality of piezoelectric transducers, a matching layer provided to the piezoelectric transducers, and a backing material that prevents propagation of ultrasonic waves from the piezoelectric transducers to the rear side. The ultrasonic probe 70 is detachably connected to the apparatus body 10. The plurality of piezoelectric transducers generate ultrasonic waves based on a driving signal supplied from ultrasonic transmitting circuitry 11 included in the apparatus body 10. The ultrasonic probe 70 may be provided with a button which is pressed in an offset process to be described later or in freezing of ultrasonic images.

Once the ultrasonic probe 70 transmits ultrasonic waves to a living body P, the transmitted ultrasonic waves are sequentially reflected by the boundary showing discontinuity of the acoustic impedance of the living tissue of the living body P, and are received as reflected wave signals by the plurality of piezoelectric transducers included in the ultrasonic probe 70. The amplitude of the received reflected wave signals depend on the difference in the acoustic impedance at the boundary showing discontinuity of the acoustic impedance that affects the reflection of ultrasonic waves. If the transmitted ultrasonic pulses are reflected in a bloodstream or on the surface of the cardiac wall, the frequency of the reflected wave signals are shifted depending on velocity components in the direction of transmitting ultrasonic waves in a moving object due to the Doppler effect. The ultrasonic probe 70 receives the reflected wave signals from the living body P, and converts the reflected wave signals into electrical signals.

The apparatus body 10 shown in FIG. 1 generates an ultrasonic image, based on reflected wave signals received by the ultrasonic probe 70. As shown in FIG. 1, the apparatus body 10 includes the ultrasonic transmitting circuitry 11, ultrasonic receiving circuitry 12, B-mode processing circuitry 13, Doppler-mode processing circuitry 14, three-dimensional processing circuitry 15, display processing circuitry 16, an internal storage circuitry 17, an image memory 18 (cine memory), an image database 19, input interface circuitry 20, communication interface circuitry 21, and control circuitry 22.

The ultrasonic transmitting circuitry 11 is a processor that supplies driving signals to the ultrasonic probe 70. The ultrasonic transmitting circuitry 11 is implemented, for example, by trigger generating circuitry, delay circuitry, pulser circuitry, etc. The trigger generating circuitry repeatedly generates rate pulses for forming transmission ultrasonic waves at a predetermined rate frequency under control of the control circuitry 22. The delay circuitry converges ultrasonic waves generated from the ultrasonic probe 70 as a beam, and applies, to each rate pulse generated by the trigger generating circuitry, a transmission delay time for each piezoelectric transducer required for determining a transmission directivity. The pulser circuitry supplies driving signals (a driving pulse) to the ultrasonic probe 70 at a timing based on the rate pulse under control of the control circuitry 22. By changing the delay time to be applied to each rate pulse from the delay circuitry, the transmission direction from the piezoelectric transducer surface can be discretionarily adjusted.

The ultrasonic receiving circuitry 12 is a processor that executes various processes on reflected wave signals received by the ultrasonic probe 70 to generate a receive signal. The ultrasonic receiving circuitry 12 is implemented, for example, by amplification circuitry, an A/D converter, reception delay circuitry, and an adder, etc. The amplification circuitry executes a gain correction process for each channel by amplifying reflected wave signals received by the ultrasonic probe 70. The A/D converter converts the gain-corrected reflected wave signals to digital signals. The reception delay circuitry delays input of the digital signals to the adder by a delay time required for determining a reception directivity. The adder adds a plurality of digital signals in which the delay time has been applied. After the addition process of the adder, receive signals are generated in which a reflected component from a direction corresponding to the reception directivity is emphasized.

The B-mode processing circuitry 13 is a processor that generates B-mode data, based on the receive signals received from the ultrasonic receiving circuitry 12. The B-mode processing circuitry 13 executes an envelope detection process and a logarithmic amplification process, etc. on the receive signals received from the ultrasonic receiving circuitry 12, and generates data (B-mode data) in which the signal intensity is expressed by the brightness intensity. The generated B-mode data is stored in a RAW data memory (not shown in the drawings) as B-mode RAW data on a two-dimensional ultrasonic scanning line.

The Doppler-mode processing circuitry 14 is a processor that generates a Doppler waveform and Doppler data, based on the receive signals received from the ultrasonic receiving circuitry 12. The Doppler-mode processing circuitry 14 extracts a blood flow signal from the receive signal, generates a Doppler waveform from the extracted blood flow signal, and generates data (Doppler data) in which information, such as a mean velocity, dispersion, power, etc. is extracted from the blood flow signal with respect to multiple points. The generated Doppler data is stored in a RAW data memory (not shown in the drawings) as Doppler RAW data on a two-dimensional ultrasonic scanning line.

The three-dimensional processing circuitry 15 is a processor that can generate volume data (three-dimensional image data) based on the data generated by the B-mode processing circuitry 13 and the Doppler-mode processing circuitry 14.

In addition, the three-dimensional processing circuitry 15 performs, to B-mode data stored in a RAW data memory, RAW-voxel conversion which includes an interpolation process taking spatial position information into consideration to generate volume data consisting of voxels in a desired range.

The three-dimensional processing circuitry 15 generates rendering image data by performing a rendering process to the generated volume data.

The display processing circuitry 16 is a processor that displays various images on the display 50. The display processing circuitry 16 generates ultrasonic image data pieces as display images by a coordinate conversion process, etc. The coordinate conversion process is a process to convert signal streams of scanning lines for ultrasonic scanning, comprising B-mode data and Doppler data, into video signals as signal streams of scanning lines in a general video format such as represented by a TV, etc. The generated ultrasonic image data piece is assigned with a predetermined body mark and at least one sample probe mark that is set on the body mark. A body mark is used to indicate, by a schematic graphic, an imaged region at the time when a subject is imaged with the ultrasonic probe. The ultrasonic image data piece assigned with a body mark and at least one sample probe mark set on the body mark, that is, the sample probe mark that defines a position where the ultrasonic probe locates on the body mark, is converted into a format based on, e.g., the DICOM (digital imaging and communication in medicine) standard, and is stored in, e.g., the image database 19.

The display processing circuitry 16 generates B-mode image data based on B-mode RAW data stored in the RAW data memory. For example, the B-mode image data contains a pixel value (brightness value) that reflects a feature of an ultrasonic probe, such as convergence of sound waves or a sound-field feature of an ultrasonic beam (for example, transmitting/receiving beam). In the B-mode image data, for example, the vicinity of a focus portion of ultrasonic waves in a region to be scanned exhibits higher brightness than that of a non-focus portion. The display processing circuitry 16 causes the display 50 to display the generated B-mode image data as an ultrasonic image.

The display processing circuitry 16 generates Doppler image data relating to an average-speed image, a dispersed image, a power image, etc., based on Doppler RAW data stored in the RAW data memory. The display processing circuitry 16 causes the display 50 to display the generated Doppler image data as an ultrasonic image.

The display processing circuitry 16 executes various processes, such as dynamic range, brightness, contrast and y curve corrections, and RGB conversion, etc., to image data piece generated in the three-dimensional processing circuitry 15, in order to convert the image data piece to a video signal. The display processing circuitry 16 causes the display 50 to display the video signal as an ultrasonic image.

The display processing circuitry 16 may generate a user interface (GUI: Graphical User Interface) through which an operator (for example, a surgeon) inputs various instructions by the input interface circuitry 20, and directs the display 50 to display the GUI. The display 50 may adopt, for example, a CRT display, a liquid crystal display, an organic EL display, an LED display, a plasma display, or any other display known in this technical field.

The internal storage circuitry 17 includes, for example, a storage medium which is readable by a processor, such as a magnetic or optical storage medium, or a semiconductor memory, etc. The internal storage circuitry 17 stores a control program for implementing ultrasonic transmission/reception, a control program for executing an image process, a control program for executing a display process, etc. The internal storage circuitry 17 stores control programs for implementing various functions according to the present embodiment. In addition, the internal storage circuitry 17 stores diagnosis information (e.g., patient ID, doctor's findings, etc.), a diagnosis protocol, a body mark generation program, and data such as a conversion table for presetting a range of color data for use in imaging, with respect to each of regions of diagnosis. The internal storage circuitry 17 may store anatomical illustrations, for example, an atlas, relating to the structures of internal organs in the body.

In addition, the internal storage circuitry 17 stores volume data and rendering image data generated by the three-dimensional processing circuitry 15, in accordance with a storing operation input via the input interface circuitry 20. In accordance with a storing operation input via the input interface circuitry 20, the internal storage circuitry 17 may store volume data and rendering image data generated by the three-dimensional processing circuitry 15, along with an operation order and an operation time. The internal storage circuitry 17 can transfer the stored data to an external device via the communication interface circuitry 21.

The internal storage circuitry 17 stores sample-model information 171 relating to a body mark. The sample-model information 171 is a collection of information (hereinafter referred to as sample model) that is standardized for each body mark so that a body mark assigned as image information to an ultrasonic image can be used as a search key for searching past images. Sample models included in the sample-model information 171 include, for example, an image data piece indicative of a shape of body mark, a position of an image according to an ultrasonic image assigned with the body mark, a direction of the image, etc. The image according to the image data piece indicative of a shape of body mark includes an image indicative of at least one probe mark set on this body mark.

Each of the position and the direction of an image is one piece of information for specifying an imaging position of an ultrasonic image. The image for specifying an imaging position is information included in incidental information assigned to an ultrasonic image data piece or/and information obtained by image recognition of an ultrasonic image based on the aforementioned ultrasonic image data piece. The information for specifying an imaging position is used as a key for searching past images based on a past image data piece in terms of whether there is a similarity in imaging position where an ultrasonic image is actually imaged.

The position of image indicates which position (region) of a subject is imaged. The position of image may be expressed by an anatomical term for a region by which an examination region can be specified, or by coordinates with a predetermined reference position as an origin. The direction of image is defined by the direction in which a subject is orientated. The direction of image is expressed by, for example, a normal vector as a unit vector indicative of the direction in which a forehead surface of a subject is oriented. The position and direction of image are included in incidental information such as a DICOM tag attached to an ultrasonic image data piece of an ultrasonic image, for example.

The internal storage circuitry 17 stores a human body model that presents an anatomical structure of a whole human body. For example, the human body model is an anatomical map containing a plurality of examination regions as structural elements, or three-dimensional image data including three-dimensional coordinate information, etc.

The image memory 18 includes, for example, a storage medium which is readable by a processor, such as a magnetic or optical storage medium, or a semiconductor memory. The image memory 18 stores image data pieces corresponding to a plurality of frames immediately before a freeze operation input through the input interface circuitry 20. The image data pieces stored in the image memory 18 are successively displayed (cine-displayed), for example.

The image database 19 stores image data pieces transferred from the external device 40. For example, the image database 19 acquires from the external device 40, an image data piece relating to a particular patient obtained by the past diagnosis, and stores the acquired image data piece. The past image data pieces include ultrasonic image data, CT (Computed Tomography) image data, MR image data, PET (Positron Emission Tomography)-CT image data, PET-MR image data, and X-ray image data. For example, the past image data pieces are stored as three-dimensional volume data and rendering image data.

The image database 19 may store a desired image data piece y reading image data stored in a storage medium such as an MO, a CD-R and a DVD.

The input interface circuitry 20 receives various instructions from an operator through the input device 60. The input device 60 is, for example, a mouse, a keyboard, a panel switch, a slider switch, a trackball, a rotary encoder, an operation panel, or a touch command screen (TCS). The input interface circuitry 20 is connected to the control circuitry 22, for example, via a bus. The input interface circuitry 20 converts an operation instruction input by the operator into electric signals, and outputs the electric signals to the control circuitry 22. In the present embodiments, the input interface circuitry 20 is not limited to interface circuitry which is connected to physical operation components such as a mouse, a keyboard, etc. The input interface circuitry 20 may include processing circuitry of electric signals which receives, as radio signals, electric signals corresponding to an operation instruction input from an external input device independent of the ultrasonic diagnostic apparatus 1, and outputs the electric signals to the control circuitry 22.

The communication interface circuitry 21 is, for example, wirelessly connected to the position sensor system 30 and receives position information transmitted from the position detecting device 32. The communication interface circuitry 21 is connected to the external device 40 through the network 100, etc., and performs data communication with the external device 40. The external device 40 is, for example, a database of a PACS (Picture Archiving and Communication System) which is a system for managing various medical image data, a database of an electronic medical record system for managing electronic medical records to which medical images are added, etc. In addition, the external device 40 may, for example, be any medical image diagnostic apparatus other than the ultrasonic diagnostic apparatus 1 according to the present embodiment, such as an X-ray CT apparatus, an MRI (Magnetic Resonance Imaging) apparatus, a nuclear medical diagnostic apparatus, an X-ray diagnostic apparatus, etc. Any standards may be applied for communication with the external device 40. For example, DICOM may be applied.

The control circuitry 22 is a processor acting as a nerve center of the ultrasonic diagnostic apparatus 1, for example. The control circuitry 22 executes the operating program stored in the internal storage circuitry 17 to realize a function corresponding to the operating program. Specifically, the control circuitry 22 includes an image recognizing function 221, a sample-model registering function 223, a mapping function 225, a similarity calculating function 227, a similar-image extracting function 229, a display controlling function 231, and a system controlling function 233.

The image recognizing function 221 is a function of performing image recognition of, e.g., an ultrasonic image assigned with predetermined incidental information as image information and obtaining necessary information. When the image recognizing function 221 is executed, the control circuitry 22 performs image recognition of an ultrasonic image assigned with a body mark by a predetermined image recognition technique. The control circuitry 22 extracts from the ultrasonic image, a region containing a shape of body mark as an image data piece indicative of a shape of body mark.

The control circuitry 22 performs image recognition with respect to a body mark region containing a probe mark region by a predetermined image recognition technique. For example, the control circuitry 22 recognizes a relative position of a probe mark with respect to a body mark. From the extracted region containing a shape of body mark, the control circuitry 22 further extracts a region containing a shape of probe mark as an image data piece indicative of a shape of probe mark. The control circuitry 22 calculates imaging information regarding an ultrasonic probe based on the recognized relative position of the probe mark with respect to the body mark, and the extracted image data piece indicative of a shape of probe mark.

The imaging information is one piece of information for specifying an imaging position of an ultrasonic image. The imaging information includes a center position of a probe, a direction of the probe, an imaging direction indicative of a direction of sliding the ultrasonic probe, etc. The center position of a probe indicates a gravity center position of the probe, which is expressed by an orthogonal coordinate system with a predetermined position of a body mark as an origin. The direction of a probe indicates, for example, the inclination of a probe mark relative to a reference which is set to a predetermined axis of the orthogonal coordinate system with a predetermined position of a body mark as an origin. The imaging direction indicates an imaging angle, for example, a flap angle, from a body surface of a subject to an imaging target inside the subject. The imaging direction is calculated from a relative direction relation between a direction of an image according to a body mark and a direction of a probe.

The sample-model registering function 223 is a function of registering in the sample-model information 171, an image data piece, which indicates a shape of body mark and is extracted by the image recognizing function 221, in association with necessary information. When the sample-model registering function 223 is executed, the control circuitry 22 associates an image data piece indicative of a shape of body mark, extracted by the image recognizing function 221, with a position and a direction of an image. The control circuitry 22 registers as a sample model, the image data piece associated with the position and direction of image, in the sample-model information 171 stored in the internal storage circuitry 17.

The mapping function 225 is a function of correlating a sample model, a probe mark, etc., with a predetermined human body model. When the mapping function 225 is executed, the control circuitry 22 correlates a sample model with a human body model stored in the internal storage circuitry 17 based on the image data piece indicative of a shape of body mark, the position of an image, and the direction of the image, which are included in the sample model. This determines a position where the sample model locates on the human body model. The position where the sample model locates on the human body model is one piece of information for specifying an imaging position of an ultrasonic image.

The control circuitry 22 correlates a probe mark with a human body model stored in the internal storage circuitry 17, based on imaging information calculated from the image data piece indicative of a shape of this probe mark. This determines a position where the probe mark locates on the human body model. The position where the probe mark locates on the human body model is one piece of information for specifying an imaging position of an ultrasonic image.

The similarity calculating function 227 is a function of calculating the similarity in ultrasonic image. When the similarity calculating function 227 is executed, the control circuitry 22 compares a position where a sample model locates on a human body model of an ultrasonic image acting as a key image for a search, and a direction of the image, with a position where a sample model locates on a human body model of a past image based on a past image data piece, and a direction of the image. The control circuitry 22 then calculates the similarity in body mark for each sample model using a predetermined evaluation function.

When the similarity calculating function 227 is executed, the control circuitry 22 compares a position where a sample model of an ultrasonic image acting as a key image for a search, calculated by the image recognizing function 221, locates on a human body model, a direction of the image, and imaging information, with a position where a sample model associated with a past image data piece locates on a human body model, a direction of the image, and imaging information. The control circuitry 22 then calculates the similarity in sample model using a predetermined evaluation function.

When the similarity calculating function 227 is executed, the control circuitry 22 compares the image feature amount based on, for example, a pixel value of an ultrasonic image, with the image feature amount based on a pixel value of a past image according to a past image data piece. The control circuitry 22 then calculates the similarity in image feature amount using a predetermined evaluation function for each past image data piece. The image feature amount includes, for example, a pixel value, a pixel value distribution, etc.

The similar-image extracting function 229 is a function of extracting a sample model or past image, etc., which satisfies a predetermined condition, based on various similarities calculated by the similarity calculating function 227. When the similar-image extracting function 229 is executed, the control circuitry 22 extracts a sample model that satisfies a predetermined condition from sample models registered in the sample-model information 171, based on the similarity in body mark. The control circuitry 22 compares a preset threshold with the similarity in body mark, for each sample model. The control circuitry 22 extracts a sample model having higher similarity in body mark than a preset threshold, from sample models registered in the sample-model information 171.

The control circuitry 22 extracts based on the similarity in sample model, a past image that satisfies a predetermined condition, from past images based on past image data pieces. The control circuitry 22 compares, for example, a preset threshold with a calculated similarity, for each past image data piece. The control circuitry 22 extracts a past image data piece having higher similarity in sample model than a preset threshold, from past images based on past image data pieces.

The control circuitry 22 further extracts based on the similarity in image feature amount, a past image that satisfies a predetermined condition, from past images based on past image data pieces. The control circuitry 22 compares, for example, a preset threshold with a calculated similarity in image feature amount, for each past image data piece. The control circuitry 22 extracts a past image data piece having higher similarity in image feature amount than a preset threshold, from past images based on past image data pieces.

The display controlling function 231 is a function of displaying a body mark or a past image as a selection candidate for an operator. When the display controlling function 231 is executed, the control circuitry 22 causes the display 50 to display a body mark based on a sample model extracted by the similar-image extracting function 229. The control circuitry 22 causes the display 50 to display a past image based on a past image data piece extracted by the similar-image extracting function 229.

The system controlling function 233 is a function of controlling basic operations, such as the input and output, at the ultrasonic diagnostic apparatus 1. When the system controlling function 233 is executed, for example, the control circuitry 22 receives selection of a body mark based on a sample model, via the input interface circuitry 20. The control circuitry 22 receives selection of a past image based on a past image data piece, via the input interface circuitry 20, for example. The control circuitry 22 reads a past image data piece corresponding to the selected past image from the image database 19.

The image recognizing function 221, the sample-model registering function 223, the mapping function 225, the similarity calculating function 227, the similar-image extracting function 229, the display controlling function 231, and the system controlling function 233 may be integrated as control programs. Alternatively, hardware circuitry specific to each function may be integrated into the control circuitry 22 itself or the apparatus body 10 as circuitry which the control circuitry 22 can refer to.

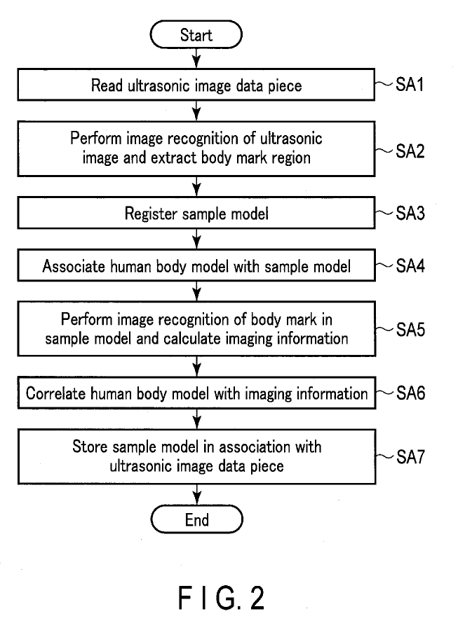

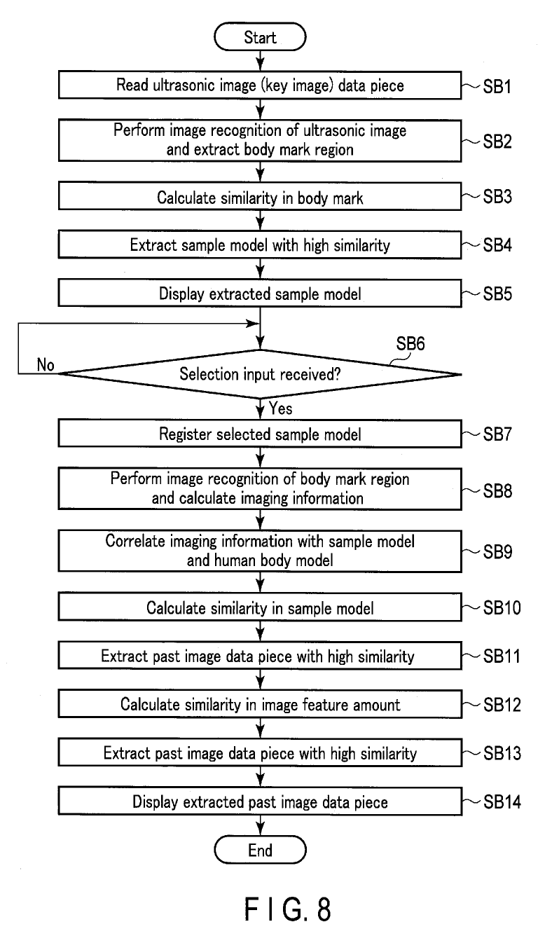

Next, the various operations of the ultrasonic diagnostic apparatus 1 according to the first embodiment will be explained with reference to the flowcharts shown in FIGS. 2 and 8.

First, the operation of associating a sample model with an ultrasonic image data piece will be described. FIG. 2 is a flowchart showing the operation of the control circuitry 22 at the time when the ultrasonic diagnostic apparatus 1 according to the first embodiment associates a sample model with an ultrasonic image data piece. The following description assumes that an ultrasonic image data piece generated by the display processing circuitry 16 is stored in the image database 19 after a body mark and at least one probe mark are preset thereto. It is also assumed that a position and a direction of an image are assigned as incidental information to an ultrasonic image data piece.

When an ultrasonic image data piece generated by the display processing circuitry 16 is stored in the image database 19, for example, the control circuitry 22 executes the image recognizing function 221 to read the stored ultrasonic image data piece (step SA1). This ultrasonic image data piece is assigned with a body mark and at least one probe mark as image information.

The control circuitry 22 executes image recognition with respect to an ultrasonic image according to the read ultrasonic image data piece. In this manner, the control circuitry 22 extracts from the ultrasonic image, a region containing a shape of body mark as image data indicative of a shape of body mark (step SA2).

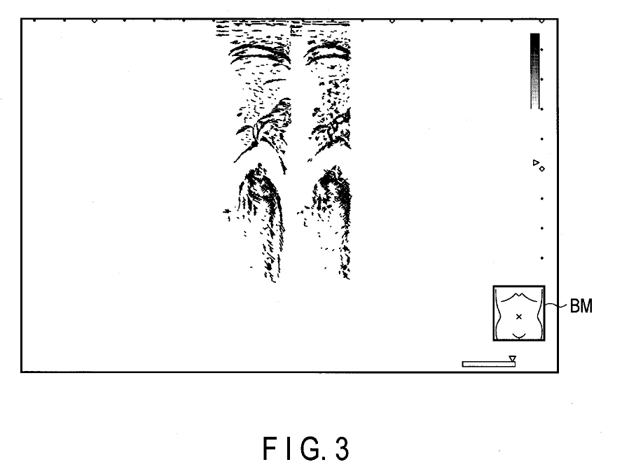

FIG. 3 is a view showing an ultrasonic image that includes a body mark region and is displayed on the display 50 according to the first embodiment. As shown in FIG. 3, the control circuitry 22 recognizes a region that includes an assembly of pixels with the maximum brightness or the minimum brightness on the ultrasonic image, and then extracts a rectangular region circumscribing this region, as a body mark region BM.

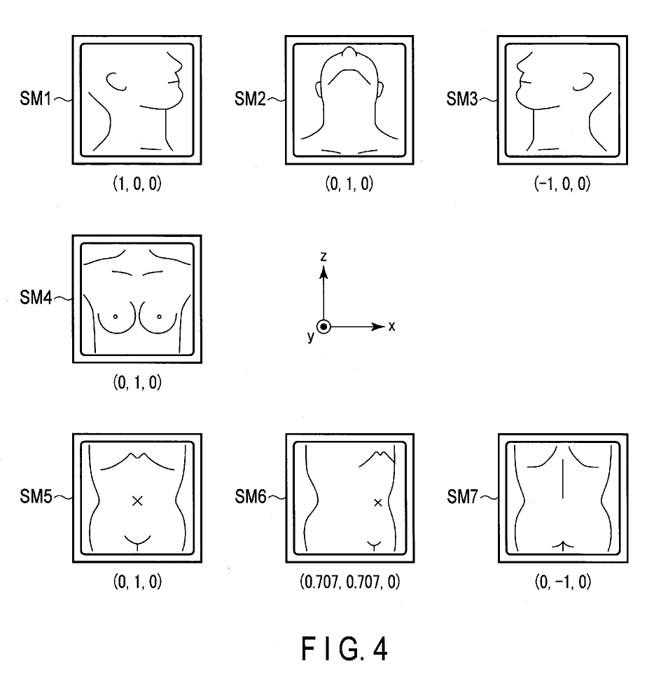

Upon extracting image data indicative of a shape of body mark from the ultrasonic image, the control circuitry 22 executes the sample-model registering function 223 to associate the extracted image data indicative of a shape of body mark with a position and a direction of an image included in incidental information assigned to the ultrasonic image. The control circuitry 22 registers the image data associated with the direction of image as a sample model in the sample-model information 171 stored in the internal storage circuitry 17 (step SA3). FIG. 4 is a view illustrating examples of a sample model registered in the sample-model information 171 according to the first embodiment. Each of an X-axis, Y-axis, and Z-axis shown in FIG. 4 is a three-dimensional orthogonal axis for indicating a direction of an image. Each of sample models shown in FIG. 4 includes an image data piece indicative of a shape of body mark, a position of an image, and a direction of the image.

According to a sample model SM1 shown in FIG. 4, the shape of body mark represents a head while the direction of image is indicated by the normal vector (1, 0, 0). According to a sample model SM2, the shape of body mark represents a head while the direction of image is indicated by the normal vector (0, 1, 0). According to a sample model SM3, the shape of body mark represents a head while the direction of image is indicated by the normal vector (−1, 0, 0). According to a sample model SM4, the shape of body mark represents a chest while the direction of image is indicated by the normal vector (0, 1, 0). According to a sample model SM5, the shape of body mark represents the lumbar region while the direction of image is indicated by the normal vector (0, 1, 0). According to a sample model SM6, the shape of body mark represents the lumbar region while the direction of image is indicated by the normal vector (0.707, 0.707, 0). According to a sample model SM 7, the shape of body mark represents the lumbar region while the direction of image is indicated by the normal vector (0, −1, 0).

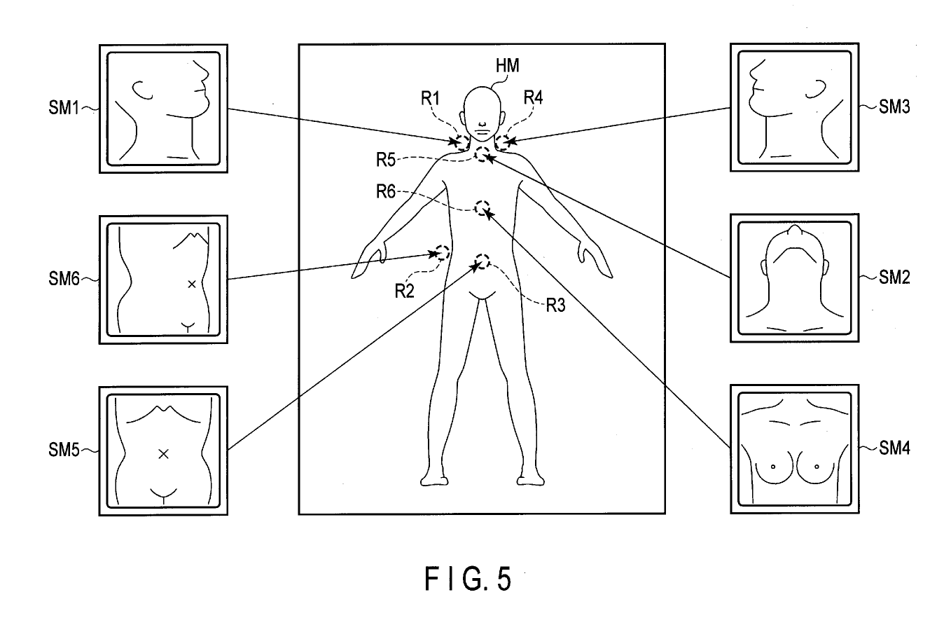

The control circuitry 22 executes the mapping function 225 to correlate a sample model with a human body model stored in the internal storage circuitry 17 based on the image data indicative of a shape of body mark, the position of an image, and the direction of the image, which are included in the sample model (step SA4). FIG. 5 is a view illustrating specifically how the ultrasonic diagnostic apparatus 1 according to the first embodiment correlates a sample model with a human body model. According to FIG. 5, the sample models are correlated with respective constituent elements indicative of anatomical regions of a human body model HM. The respective anatomical regions include direction elements. Specifically, regions R1, R2, R3, R4, R5, and R6 on the human body model HM represent, for example, the right side face of the head, the diagonally right side face of the lumbar region, the front face of the lumbar region, the left side face of the head, the front face of the head, and the front face of the chest, respectively. The control circuitry 22 then correlates the regions R1, R2, R3, R4, R5, and R6 on the human body model HM with the sample models SM1, SM6, SM5, SM3, SM2, and SM4 shown in FIG. 4, respectively. This determines positions where the sample models locate on the human body model HM.

The control circuitry 22 executes the image recognizing function 221 to perform image recognition with respect to the body mark region by a predetermined image recognition technique. Specifically, the control circuitry 22 performs image recognition of the image data indicative of the shape of body mark included in the sample model registered in the sample-model information 171 in step SA3, and recognizes a relative position of the probe mark with respect to the body mark. The control circuitry 22 extracts a region including a shape of probe mark as an image data piece indicative of a shape of probe mark, from the image data piece indicative of the shape of body mark included in the sample model registered in the sample-model information 171 in step SA3. The control circuitry 22 calculates imaging information regarding an ultrasonic probe based on the relative position of the probe mark with respect to the body mark and the extracted image data piece indicative of the shape of probe mark (step SA5). The control circuitry 22 associates a probe mark with a sample model based on the calculated imaging information.

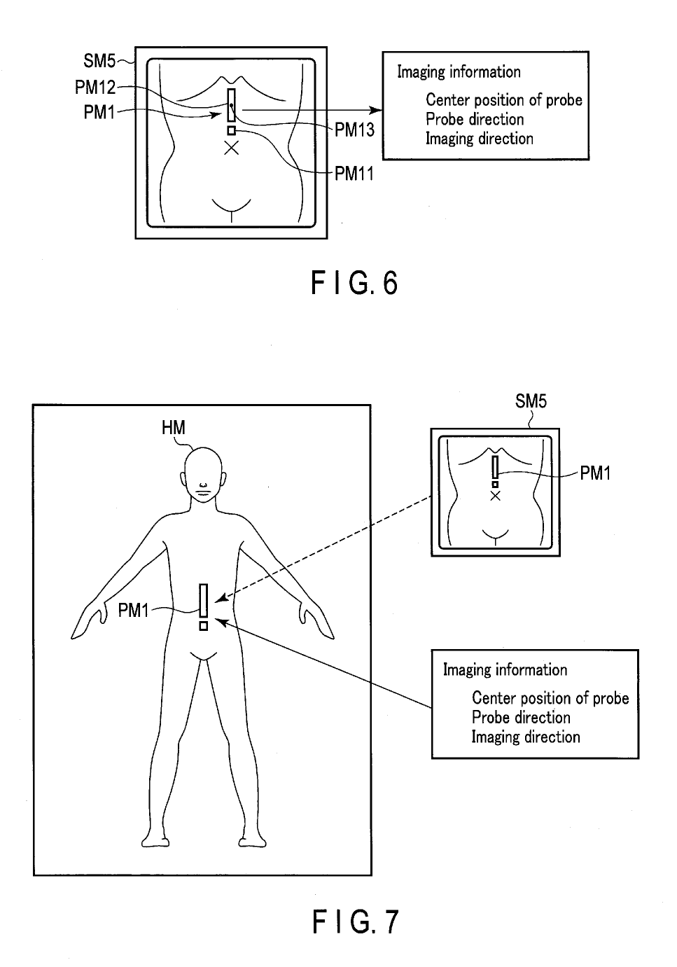

FIG. 6 is a view illustrating specifically how the ultrasonic diagnostic apparatus 1 according to the first embodiment calculates imaging information based on a shape of probe mark included in a body mark of a sample model. According to FIG. 6, the body mark of the sample model SM5 shown in FIG. 4 includes a probe mark PM1. The probe mark PM1 represents a linear-type probe and includes a region PM11 in a square shape and a region PM12 in a rectangular shape. The region PM11 represents, for example, the starting edge of an electric scanning direction. The region PM12 represents, for example, a scanning surface.

The control circuitry 22 calculates based on, e.g., a shape of a body mark according to the sample model SM5 and a shape of the region PM12, a center position PM13 of a probe represented by the orthogonal coordinate system with a predetermined position of this body mark as an origin. The control circuitry 22 calculates based on, e.g., the shape of a body mark according to the sample model SM5 and the shape of the region PM12, the inclination of a probe mark relative to a reference which is set to a predetermined axis of the orthogonal coordinate system with a predetermined position of this body mark as an origin. Based on a relative direction relation between the direction of the image of the body mark according to the sample model SM5 and the direction of the probe, the control circuitry 22 calculates as the imaging direction, the inclination relative to a reference which is set to a predetermined surface of the orthogonal coordinate system with a predetermined position of this body mark as an origin.

The control circuitry 22 may change how to calculate imaging information depending on the shape of a probe mark, such as a sector-type probe mark having a fan-like basic shape, a convex-type probe mark having an arch-like basic shape, etc.

The control circuitry 22 executes the mapping function 225 to correlate the probe mark according to the calculated imaging information with the human body model (step SA6). Specifically, the control circuitry 22 converts the coordinate system of the imaging information into the coordinate system of the human body model. FIG. 7 is a view illustrating specifically how the ultrasonic diagnostic apparatus 1 according to the first embodiment correlates a probe mark with a human body model. According to FIG. 7, the probe mark PM1 included on the body mark image of the sample model SM5 shown in FIG. 4 is correlated with the human model HM based on the calculated imaging information and the position of the sample model SM5 on the human body model. The control circuitry 22 correlates the probe mark with the human body model by, for example, converting the coordinate system of the imaging information calculated in step SA5, into the coordinate system specified by the human body model.

Upon correlating the human body model with the sample model and the probe mark of this sample model, the control circuitry 22 stores as past image data, this sample model in association with an ultrasonic image data piece in the image database 19 (step SA7).

Next, the operation of searching past image data will be described. FIG. 8 is a flowchart showing the operation of the control circuitry when the ultrasonic diagnostic apparatus according to the first embodiment searches for a past image similar to an ultrasonic image of an obtained ultrasonic image data piece.

The following description assumes that a past image data piece is associated with a sample model which is correlated with a human body model. It is also assumed that both a position and a direction of an image are assigned in advance as incidental information to an ultrasonic image data piece generated by the display processing circuitry 16. It is also assumed that a sample model is not associated with an ultrasonic image data piece of an ultrasonic image as a key image for a search.

When an ultrasonic image data piece generated by the display processing circuitry 16 is stored in the image database 19 before or during an operation, for example, the control circuitry 22 executes the image recognizing function 221 to read the stored ultrasonic image data piece (step SB1).

The control circuitry 22 executes image recognition with respect to an ultrasonic image according to the read ultrasonic image data piece. In this manner, the control circuitry 22 extracts from the ultrasonic image, a region containing a shape of body mark as image data indicative of a shape of body mark (step SB2).

The control circuitry 22 executes the similarity calculating function 227 to extract a position and a direction of an image from incidental information of the read ultrasonic image data piece. The control circuitry 22 compares the extracted position and direction of image and the image data piece indicative of the shape of body mark extracted in step SB2, with the position and direction of image included in the sample model registered in the sample-model information 171 and the image data piece indicative of the shape of body mark, thereby calculating the similarity in body mark using a predetermined evaluation function for each sample model (step SB3). The similarity is represented by values from 0 to 1, and a value closer to 1 represents a higher similarity.

If a direction of image extracted from incidental information of an ultrasonic image is different from a direction of an image included in a sample model, the control circuitry 22 may make the direction of the image extracted from the incidental information of the ultrasonic image conform to the direction of the image included in the sample model. When doing this, the control circuitry 22 changes the shape of body mark extracted from the ultrasonic image in accordance with the change in the direction of image, for example. The control circuitry 22 calculates the similarity in body mark by comparing the changed direction of image and the image data indicative of the shape of body mark, with the direction of image included in the sample model registered in the sample-model information 171 and the image data indicative of the shape of body mark.

The control circuitry 22 executes the similar-image extracting function 229 to compare a preset threshold with the similarity in body mark, for each sample model. The control circuitry 22 extracts a sample model having a higher similarity in body mark than a preset threshold, from sample models registered in the sample-model information 171 (step SB4). For example, a preset threshold is 0.8.

The control circuitry 22 executes the display controlling function 231 to cause the display 50 to display a body mark based on the sample model extracted in step SB4 (step SB5).

Upon displaying the extracted sample models, the control circuitry 22 receives selection input for selecting a predetermined sample model from among the displayed sample models, via the input interface circuitry 20 (step SB6).

When selection input for selecting a predetermined sample model is made, the control circuitry 22 registers the selected sample model in the sample-model information 171 (step SB7). The control circuitry 22 associates the selected sample model with the ultrasonic image data piece read in step SB1.

The control circuitry 22 executes the image recognizing function 221 to perform image recognition with respect to the body mark region of the sample model associated with the ultrasonic image data piece in step SB7, by a predetermined image recognition technique. Specifically, the control circuitry 22 performs image recognition with respect to the image data piece indicative of the shape of body mark included in the sample model, and recognizes a relative position of the probe mark with respect to the body mark. The control circuitry 22 extracts a region including a shape of probe mark as an image data piece indicative of a shape of probe mark, from the image data piece indicative of the shape of body mark included in the sample model. The control circuitry 22 calculates imaging information regarding an ultrasonic probe based on the relative position of the probe mark with respect to the body mark and the extracted image data piece indicative of the shape of probe mark (step SB8). As described in connection with step SA5 shown in FIG. 2, the control circuitry 22 may change how to calculate imaging information depending on the shape of a probe mark.

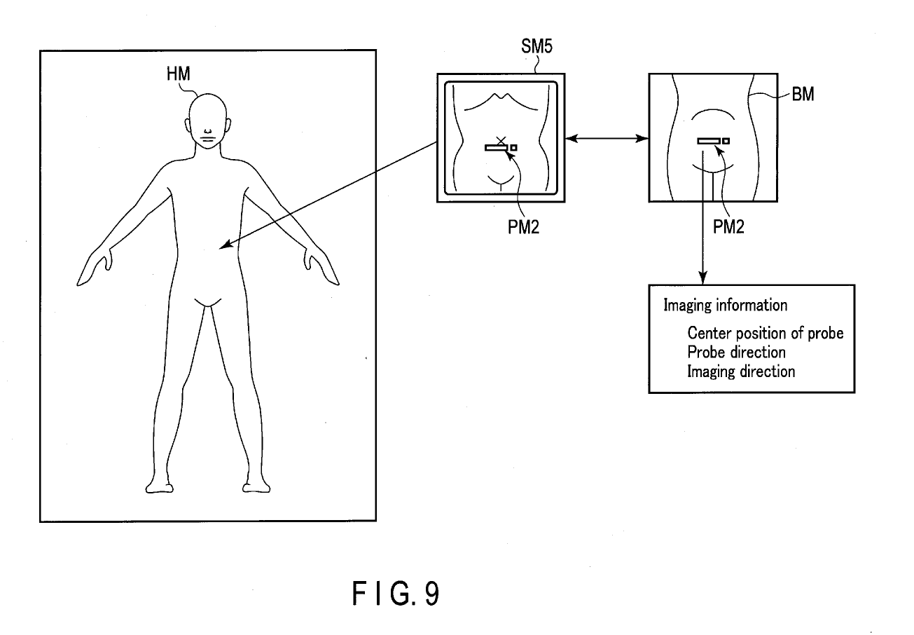

The control circuitry 22 executes the mapping function 225 to correlate a probe mark of the calculated imaging information with the sample model selected in step SB6. The control circuitry 22 correlates the imaging information correlated with the sample model with the human body model stored in the internal storage circuitry 17 (step SB9). FIG. 9 is a view illustrating specifically how the ultrasonic diagnostic apparatus 1 according to the first embodiment correlates a probe mark with a sample model and a human body model. According to FIG. 9, the control circuitry 22 converts the coordinate system of the imaging information according to the probe mark PM2 included in the body mark region BM on the ultrasonic image into the coordinate system specified by the sample model SM5. In this manner, the probe mark PM2 is correlated with the sample model SM5. The control circuitry 22 further converts the imaging information converted into the coordinate system specified by the sample model SM5, into the coordinate system specified by the human body model HM. In this manner, the probe mark PM2 is correlated with the human body model HM.

The control circuitry 22 executes the similarity calculating function 227 to compare the sample model and imaging information according to the ultrasonic image data piece read in step SB1, with a sample model and imaging information according to a past image data piece, thereby calculating the similarity for each past image (step SB10). Specifically, the control circuitry 22 compares a position where a sample model of an ultrasonic image based on the read ultrasonic image data piece locates on a human body model, a direction of the image, and imaging information, with a position where a sample model associated with past image data locates on a human body model, a direction of the image, and imaging information. The control circuitry 22 then calculates the similarity in sample model using a predetermined evaluation function.

The control circuitry 22 executes the similar-image extracting function 229 to compare the similarity in the calculated sample model with a preset threshold for each past image data piece. The control circuitry 22 extracts a past image data piece having a higher similarity in sample model than a preset threshold, from past images based on past image data pieces (step SB11). For example, a preset threshold is 0.8.

The control circuitry 22 executes the similarity calculating function 227 to compare an image feature amount based on, for example, the ultrasonic image data piece read in step SB1, with an image feature amount based on a pixel value of a past image according to a past image data piece. The control circuitry 22 then calculates the similarity in image feature amount using a predetermined evaluation function for each past image data piece (step SB12). When the control circuitry 22 makes a comparison of image feature amount based on a pixel value, it is preferable that a comparison target be set to a region other than a lesion region which is prone to change in shape.

The control circuitry 22 executes the similar-image extracting function 229 to compare a preset threshold with the calculated similarity in image feature amount, for each past image data piece. The control circuitry 22 extracts a past image data piece having a higher similarity in image feature amount than a preset threshold, from past images based on past image data (step SB13).

The control circuitry 22 executes the display controlling function 231 to cause the display 50 to display a past image based on the past image data piece extracted in step SB13 (step SB14).

According to the first embodiment, the control circuitry 22 extracts a position of an image and a direction of the image from incidental information of an ultrasonic image data piece of an ultrasonic image as a key image for a search, assigned with a body mark and a probe mark. The control circuitry 22 performs image recognition with respect to an ultrasonic image assigned with a body mark and a probe mark, and calculates imaging information such as a center position of a probe, a direction of the probe, an imaging direction indicative of a direction of sliding the ultrasonic probe, etc. Specifically, the control circuitry 22 compares a position where a sample model of an ultrasonic image as a key image for a search locates on a human body model, a direction of the image, and imaging information, with a position where a sample model added to a past image data piece locates on a human body model, a direction of the image, and imaging information. The control circuitry 22 then calculates the similarity in sample model using a predetermined evaluation function. The control circuitry 22 extracts based on the similarity in sample model, a past image that satisfies a predetermined condition, from past images according to past image data pieces. The control circuitry 22 causes the display 50 to display a past image based on the extracted past image data piece.

This enables past image data to be searched under searching conditions which consider an actual imaging position. The calculation to make a comparison of information for specifying an imaging position incurs less load than the calculation to make a comparison of image feature amount such as a pixel value.

Accordingly, the ultrasonic diagnostic apparatus according to the first embodiment enables the accurate and efficient search for a past image that is similar to an ultrasonic image in the vicinity of a disease site as a target.

According to the first embodiment, the control circuitry 22 performs a search based on the calculation of similarity in sample model, whose load is relatively light. After limiting past image data as a search target by this search, the control circuitry 22 performs a search based on the calculation of similarity in image feature amount, whose load is relatively heavy. This improves the efficiency of a search as compared to a search based only on the calculation of similarity in image feature amount.

### Second Embodiment

The first embodiment has been described based on the case of performing image recognition with respect to an ultrasonic image as a key image for a search, obtaining information regarding a body mark and a sample probe mark as comparison targets, and searching for a past image data piece using this information. The second embodiment will be described based on the case of causing an operator, etc., to make a selection from probe marks correlated with a human body model, obtaining imaging information associated with the selected probe mark, and searching for a past image data piece using this imaging information.

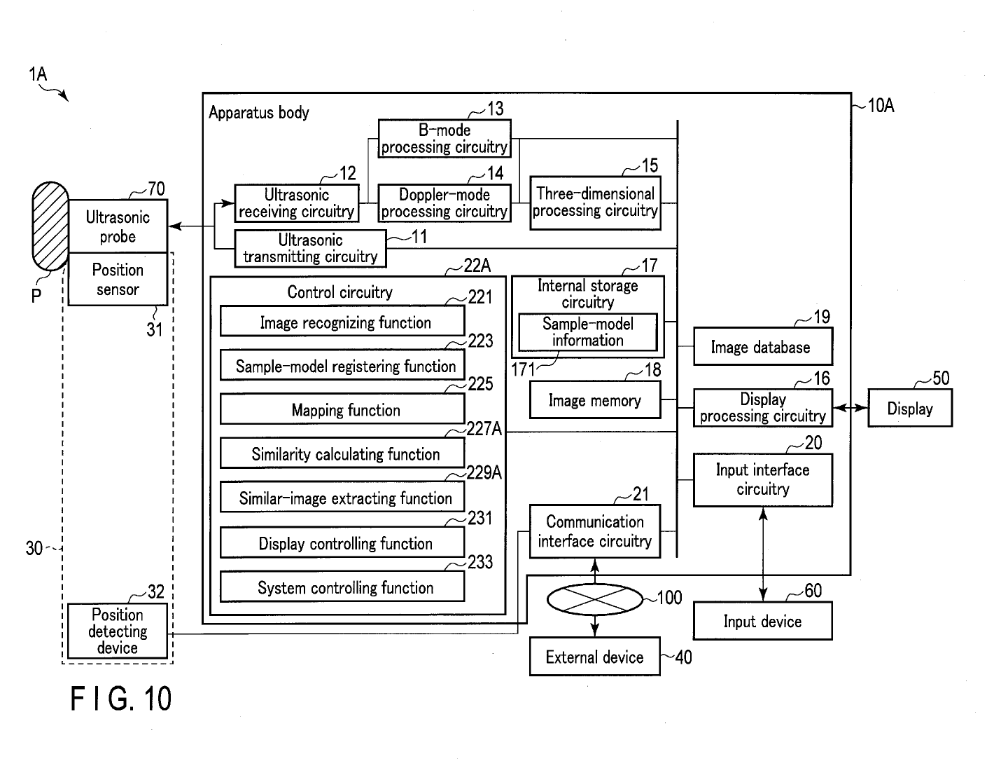

An ultrasonic diagnostic apparatus 1A according to the second embodiment will be described with reference to the block diagram shown in FIG. 10.

As shown in FIG. 10, the ultrasonic diagnostic apparatus 1A includes an apparatus body 10A, the ultrasonic probe 70, the position sensor system 30, the display 50, and the input device 60. The apparatus body 10A is connected to the external device 40 via the network 100. The apparatus body 10A is also connected to the position sensor system 30, the display 50, and the input device 60.

The apparatus body 10A shown in FIG. 10 generates an ultrasonic image, based on reflected wave signals received by the ultrasonic probe 70. As shown in FIG. 10, the apparatus body 10A includes the ultrasonic transmitting circuitry 11, the ultrasonic receiving circuitry 12, the B-mode processing circuitry 13, the Doppler-mode processing circuitry 14, the three-dimensional processing circuitry 15, the display processing circuitry 16, the internal storage circuitry 17, the image memory 18 (cine memory), the image database 19, the input interface circuitry 20, the communication interface circuitry 21, and control circuitry 22A.

The control circuitry 22A is a processor acting as a nerve center of the ultrasonic diagnostic apparatus 1A, for example. The control circuitry 22A executes the operating program stored in the internal storage circuitry 17 to realize a function corresponding to the operating program. Specifically, the control circuitry 22A includes the image recognizing function 221, the sample-model registering function 223, the mapping function 225, a similarity calculating function 227A, a similar-image extracting function 229A, the display controlling function 231, and the system controlling function 233.

In addition to the functions included in the similarity calculating function 227, the similarity calculating function 227A further includes a function of calculating a similarity in probe mark by comparing a predetermined probe mark with a probe mark set in a past image data piece. When the similarity calculating function 227A is executed, the control circuitry 22A compares imaging information associated with a probe mark correlated with a human body model, with imaging information associated with a probe mark set in a past image data piece, thereby calculating the similarity in probe mark using a predetermined evaluation function.

The similar-image extracting function 229A is a function of extracting a past image, etc., which satisfies a predetermined condition, based on the similarity in probe mark, calculated by the similarity calculating function 227A. When the similar-image extracting function 229A is executed, the control circuitry 22A extracts a past image which satisfies a predetermined condition from past images according to past image data pieces. The control circuitry 22A compares, for example, a preset threshold with the similarity in probe mark, calculated for each of probe marks according to past image data pieces. The control circuitry 22A extracts a past image data piece whose calculated similarity is higher than a preset threshold value.

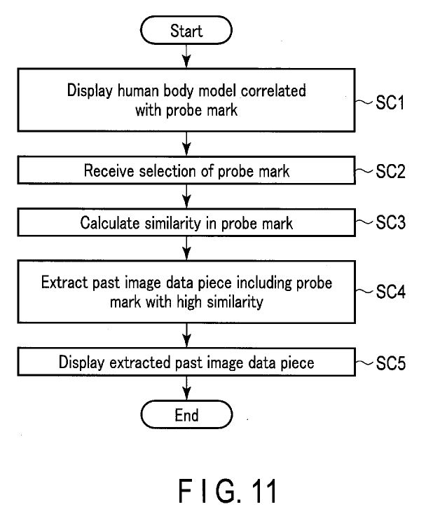

The operation of the ultrasonic diagnostic apparatus 1A according to the second embodiment will be described with reference to the flowchart shown in FIG. 11.

FIG. 11 is a flowchart showing the operation of the control circuitry 22A at the time when the ultrasonic diagnostic apparatus 1A according to the second embodiment searches for a past image having a sample probe mark similar to a designated sample probe mark. The following description assumes that a human body model is correlated with a plurality of probe marks in advance. It is also assumed that each of probe marks is associated with imaging information that is correlated with a human body model, that is, imaging information converted into the coordinate system specified by the human body model.

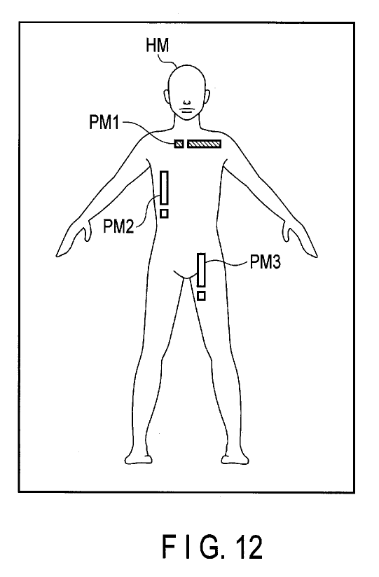

The control circuitry 22A executes the display controlling function 231 to cause the display 50 to display a human body model correlated with a plurality of probe marks (step SC1). FIG. 12 is a view showing a human body model displayed on the display 50 according to the second embodiment. FIG. 12 shows the human body model HM on which probe marks PM1, PM2, and PM3 are displayed.

The control circuitry 22A receives selection of probe marks on the human body model displayed on the display 50, via the input interface circuitry 20 (step SC2). Specifically, the control circuitry 22A receives selection of any one of the probe marks PM1, PM2, and PM3 shown in FIG. 12. The following description assumes that the probe mark PM1 is designated.

When the probe mark PM1 shown in FIG. 12 is designated, the control circuitry 22A calculates the similarity between the designated probe mark PM1 and a probe mark according to a past image data piece (step SC3). Specifically, the control circuitry 22A compares imaging information associated with the probe mark. PM1 with imaging information associated with the probe mark according to a past image data piece using a predetermined evaluation function for each past image data piece, thereby calculating the similarity in probe mark.

The control circuitry 22A executes the similar-image extracting function 229A to compare, for example, a preset threshold with the similarity in probe mark, calculated for each past image data piece. The control circuitry 22A extracts a past image data piece whose calculated similarity in probe mark is higher than a preset threshold value (step SC4).

The control circuitry 22A executes the display controlling function 231 to cause the display 50 to display a past image according to the past image data piece extracted in step SC4 (step SC5).

According to the second embodiment, the control circuitry 22A uses as a search key, imaging information associated with a probe mark selected on a human body model, and extracts a past image data piece that has similar imaging information. Accordingly, a past image data piece can be searched directly from a probe mark. This broadens a variation of search for a past image that is similar to an ultrasonic image in the vicinity of a disease site as a target.

Described in the second embodiment is the case where an operator, etc., is caused to select one of probe marks correlated with a human body model and search for a past image data piece in which a probe mark similar to the selected probe mark is set. However, this is not exhaustive. For example, an operator, etc., may be caused to select one of probe marks correlated with a body mark and search for a past image data piece in which a probe mark similar to the selected probe mark is set.

### Third Embodiment

The first embodiment has been described based on the case of performing image recognition with respect to an ultrasonic image as a key image for a search, obtaining information regarding a body mark and a sample probe mark as comparison targets, and searching for a past image data piece using this information. The third embodiment will be described based on the case of performing image recognition with respect to an ultrasonic image as a key image for a search, obtaining information regarding an annotation as a comparison target, and searching for a past image data piece using this information.

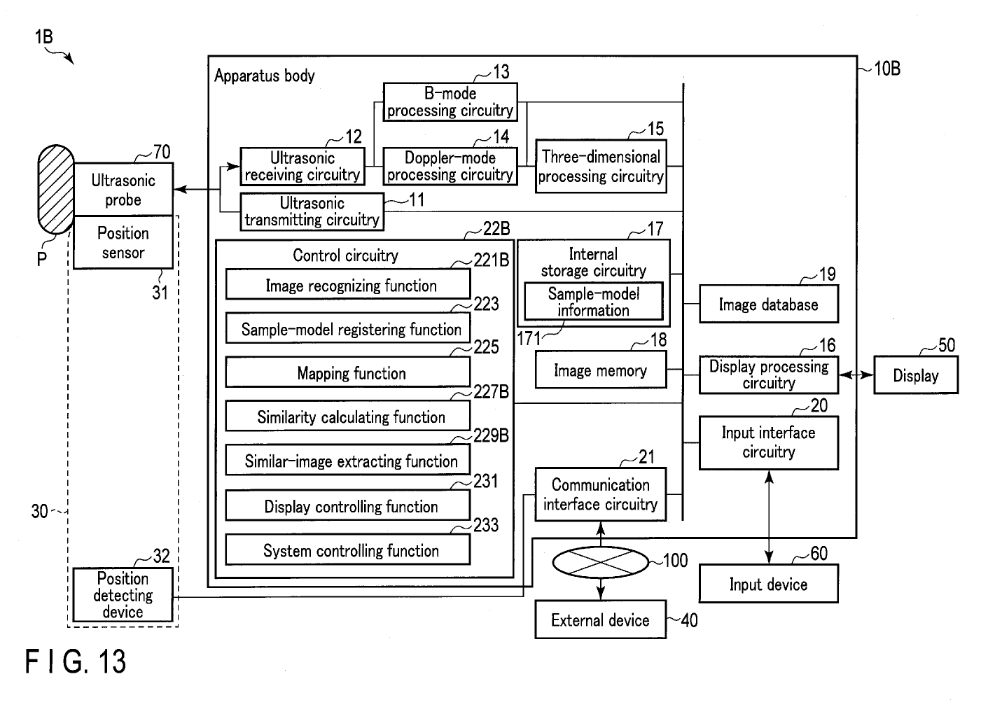

An ultrasonic diagnostic apparatus 1B according to the third embodiment will be described with reference to the block diagram shown in FIG. 13.

As shown in FIG. 13, the ultrasonic diagnostic apparatus 1B includes an apparatus body 10B, the ultrasonic probe 70, the position sensor system 30, the display 50, and the input device 60. The apparatus body 10A is connected to the external device 40 via the network 100. The apparatus body 10B is also connected to the position sensor system 30, the display 50, and the input device 60.

The apparatus body 10B shown in FIG. 13 generates an ultrasonic image, based on reflected wave signals received by the ultrasonic probe 70. As shown in FIG. 13, the apparatus body 10B includes the ultrasonic transmitting circuitry 11, the ultrasonic receiving circuitry 12, the B-mode processing circuitry 13, the Doppler-mode processing circuitry 14, the three-dimensional processing circuitry 15, the display processing circuitry 16, the internal storage circuitry 17, the image memory 18 (cine memory), the image database 19, the input interface circuitry 20, the communication interface circuitry 21, and control circuitry 22B.

The control circuitry 22B is a processor acting as a nerve center of the ultrasonic diagnostic apparatus 1B, for example. The control circuitry 22B executes the operating program stored in the internal storage circuitry 17 to realize a function corresponding to the operating program. Specifically, the control circuitry 22B includes an image recognizing function 221B, the sample-model registering function 223, the mapping function 225, a similarity calculating function 227B, a similar-image extracting function 229B, the display controlling function 231, and the system controlling function 233.

In addition to the functions included in the image recognizing function 221, the image recognizing function 221B further includes a function of performing image recognition with respect to an ultrasonic image assigned with an annotation. When the image recognizing function 221B is executed, the control circuitry 22B performs image recognition of an ultrasonic image assigned with a predetermined annotation by a predetermined image recognition technique. The control circuitry 22B extracts an annotation region from the ultrasonic image. Specifically, the control circuitry 22B recognizes a region which includes an assembly of pixels of maximum brightness or minimum brightness on the ultrasonic image, and then extracts a rectangular region circumscribing this region, as an annotation region.

In addition to the functions included in the similarity calculating function 227, the similarity calculating function 227B further includes a function of calculating the similarity by comparing an annotation assigned to an ultrasonic image with an annotation assigned to a past image data piece. When the similarity calculating function 227B is executed, the control circuitry 22B extracts a character string from the annotation region extracted from the ultrasonic image. The extracted character string is one piece of information for specifying an imaging position. Examples of a character string include a character string indicative of the right and left sides of a subject, a character string indicative of a region corresponding to a subject, etc. The control circuitry 22 compares the extracted character string with a character string included in an annotation assigned to a past image data piece, thereby calculating the similarity in annotation by using a predetermined evaluation function.

The similar-image extracting function 229B is a function of extracting a past image, etc., which satisfies a predetermined condition, based on the similarity calculated by the similarity calculating function 227B. When the similar-image extracting function 229B is executed, the control circuitry 22B extracts a past image which satisfies a predetermined condition from past images according to past image data pieces. The control circuitry 22B compares, for example, a preset threshold with the similarity in annotation, calculated for each of annotations according to past image data pieces. The control circuitry 22A extracts a past image data piece whose calculated similarity is higher than a preset threshold value.

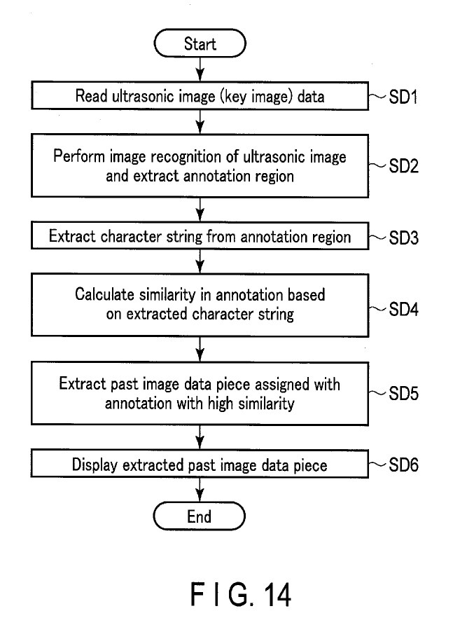

Next, the operation of the ultrasonic diagnostic apparatus 1B according to the third embodiment will be described with reference to the flowchart shown in FIG. 14.

FIG. 14 is a flowchart showing the operation of the control circuitry 22B at the time when the ultrasonic diagnostic apparatus 1B according to the third embodiment searches for a past image similar to an ultrasonic image of an obtained ultrasonic image data piece.

The following description assumes that an annotation is assigned in advance as image information to a past image data piece. It is also assumed that image recognition is performed in advance with respect to a past image according to a past image data piece to extract a character string pertaining to an annotation assigned to this past image data piece. In addition, the extracted character string is assumed to be associated with the past image data piece. Furthermore, an annotation is assigned in advance as image information to an ultrasonic image data piece acting as a key image for a search; however, image recognition is not performed with respect to this data piece.

When an annotation is assigned to an ultrasonic image data piece generated by the image processing circuitry 16 and this data piece is stored in the image database 19 before or during an operation, for example, the control circuitry 22B executes the image recognizing function 221B to read the stored ultrasonic image data piece (step SD1). This ultrasonic image data piece has an annotation assigned as image information.

The control circuitry 22B executes image recognition with respect to an ultrasonic image of the obtained ultrasonic image data piece. In this manner, the control circuitry 22B extracts an annotation region from the ultrasonic image (step SD2).

The control circuitry 22B executes the similarity calculating function 227B to extract a character string from the extracted annotation region (SD3). The control circuitry 22B compares the extracted character string with a character string included in an annotation region assigned to a past image data piece, thereby calculating the similarity in annotation by using a predetermined evaluation function (step SD4). The control circuitry 22B may add a position of annotation on an ultrasonic image as an element for use in the calculation of similarity in annotation. In doing so, the control circuitry 22B assumes that a lesion region locates in the center, and sets the similarity in annotation in a manner so that the similarity is higher as a character string locates closer to the center of an ultrasonic image. This enables extraction of a past image data piece whose observation is more desired by an operator, etc.

The control circuitry 22B executes the similar-image extracting function 229B to compare, for example, a preset threshold with the similarity in annotation, calculated for each of past image data pieces. The control circuitry 22B extracts a past image data piece assigned with an annotation whose calculated similarity in annotation is higher than a preset threshold value (step SD5).

The control circuitry 22B executes the display controlling function 231 to cause the display 50 to display a past image based on the past image data piece extracted in step SD5 (step SD6).

According to the third embodiment, the control circuitry 22B performs image recognition with respect to an annotation included in an ultrasonic image data piece acting as a key image for a search, and extracts an annotation region. The control circuitry 22B extracts a character string from the extracted annotation region. The control circuitry 22B searches for a past image data piece using the extracted character string as a search key. This broadens a variation of search for a past image that is similar to an ultrasonic image in the vicinity of a disease site as a target.

Described in the third embodiment is the case where the control circuitry 22B performs image recognition with respect to an ultrasonic image based on an ultrasonic image data piece stored in the image database 19, and extracts a character string from an annotation region assigned to this ultrasonic image. However, this is not exhaustive. The control circuitry 22B may search for a past image data piece using a character string designated by an operator, etc., as a search key.

In the third embodiment, the control circuitry 22B may correlate an annotation with a human body model based on a position of this annotation on an ultrasonic image. In making this correlation, the control circuitry 22B compares a position of annotation on a human body model and a character string extracted from an annotation region, thereby calculating the similarity in annotation using a predetermined evaluation function. The position of annotation is one piece of information for specifying an imaging position of an ultrasonic image.

In the third embodiment, for example, the control circuitry 22B may cause the image database 19 to store a character string obtained as a result of image recognition of an ultrasonic image assigned with an annotation, and a position of the annotation, in association with this ultrasonic image data piece.

### Other Embodiments

In the embodiments described above, the control circuitry 22 extracts an image data piece indicative of a shape of body mark from an ultrasonic image, and correlates the extracted image data piece indicative of a shape of body mark with a position and a direction of an image included in incidental information assigned to the ultrasonic image. However, a direction of image to be associated may be specified as information with a wide range, such as a range including the direction of image. In step SA3 shown in FIG. 2, for example, the control circuitry 22 analyzes the image data piece indicative of a shape of body mark extracted in step SA2, and estimates a range including a direction of image. Specifically, the control circuitry 22 makes an estimation based on, for example, the image data piece indicative of the shape of body mark included in the sample model SM6 shown in FIG. 4, and estimates a range including a direction of image between the normal vector (0.500, 0.866, 0) and the normal vector (0.866, 0.500, 0). The control circuitry 22 correlates the image data piece indicative of the shape of body mark with the range including the position of image included in the incidental information assigned to the ultrasonic image and the estimated range including the direction of image. The control circuitry 22 registers as a sample model, the image data piece associated with the position of image and the range including the direction of image in the sample-model information 171 stored in the internal storage circuitry 17.

In the embodiments described above, the control circuitry 22 calculates imaging information regarding an ultrasonic probe, in particular, a center position of a probe, a direction of the probe, an imaging direction, etc., based on a relative position of a probe mark included in an extracted body mark, and an extracted image data piece indicative of a shape of the probe mark. However, a center position of a probe, a direction of the probe, an imaging direction, etc., may be specified as information with a wide range; for example, a range including a center position of a probe, a range including a direction of the probe, a range including an imaging direction, etc. That is, in step SA5 shown in FIG. 2 or step SB8 shown in FIG. 8, for example, the control circuitry 22 estimates imaging information regarding an ultrasonic probe, in particular, a range including a center position of a probe, a range including a direction of the probe, a range including an imaging direction, etc., from a relative position of a probe mark with respect to a body mark, and an extracted image data piece indicative of a shape of the probe mark.

In making this estimation, for example, the control circuitry 22 estimates a range including a center position of the probe, a range including a direction of the probe, a range including imaging direction, etc., in accordance with a type of body mark. Examples of a type of body mark include “thyroid”, “breast”, “heat”, etc. In the case where a type of body mark is “thyroid”, for example, how to apply the ultrasonic probe to a subject is limited due to a location of the thyroid region and its characteristics. In this case, therefore, the estimated ranges including a center position of a probe, a direction of the probe, and imaging direction are relatively narrow. In the case where a type of body mark is “breast”, for example, how to apply the ultrasonic probe to a subject is relatively unlimited due to a location of the heart region and its characteristics. In this case, therefore, the estimated ranges including a center position of a probe, a direction of the probe, and imaging direction are relatively wide. In the case where a type of body mark is “heart”, for example, how to apply the ultrasonic probe to a subject is extremely limited due to a location of the heart region and its characteristics. In this case, therefore, the estimated ranges including a center position of a probe, a direction of the probe, and imaging direction are very narrow.

In step SB10 shown in FIG. 8, for example, the control circuitry 22 compares for each of past images, a sample model including an estimated range of a direction of an image according to an ultrasonic image data piece read in step SB1 shown in FIG. 8, and imaging information, with a sample model including an estimated range including a direction of an image according to a past image data piece, and imaging information, thereby calculating the similarity for each of past images. In making this calculation, for example, the control circuitry 22 compares a range including a position and a direction of an image according to an ultrasonic image data piece, with a range including a position and a direction of an image according to a past image data piece, thereby calculating the similarity in body mark based on a position relation between these images of both data pieces and the degree of overlapping between the ranges including the directions of the images.

For example, the control circuitry 22 compares a range including a position and a direction of an image according to an ultrasonic image data piece, a range including a center position of a probe included in imaging information, a range including a direction of the probe, and a range including an imaging direction, with a range including a position and a direction of an image according to a past image data piece, a range including a center position of a probe included in imaging information, a range including a direction of the probe, and a range including an imaging direction. The control circuitry 22 then calculates the similarity in sample model based on a position relation between these images of both image data pieces, the degree of overlapping between the ranges including the directions of the images, the degree of overlapping between the scopes including the center positions of the probe, the degree of overlapping between the ranges including the directions of the probe, and the degree of overlapping between the scopes including the imaging directions.

For example, the control circuitry 22 calculates the similarity based on a sum of a position relation between the images of both image data pieces, the degree of overlapping between the ranges including the directions of these images, the degree of overlapping between the ranges including the center positions of probe, the degree of overlapping between the ranges including the directions of the probe, and the degree of overlapping between the ranges including the imaging directions. For example, the control circuitry 22 may calculate the similarity using at least one of the degree of overlapping between the ranges including the directions of the images, the degree of overlapping between the center positions of the probe, the degree of overlapping between the scopes including the directions of the probe, and the degree of overlapping between the scopes including the imaging directions.

According to the embodiments described above, the image recognition of a body mark, a probe mark, and/or an annotation is performed with respect to both of an ultrasonic image as a key image for a search and a past image according to a past image data piece. However, this is not exhaustive. That is, the image recognition may be performed to at least one of an ultrasonic image as a key image for a search and a past image according to a past image data piece. For example, the control circuitry 22 according to the first embodiment calculates information for specifying an imaging position of a past image by performing the image recognition of a body mark and a probe mark. However, the control circuitry 22 does not perform the image recognition with respect to an ultrasonic image as a key image for a search. Instead, the control circuitry 22 calculates information for specifying an imaging position of an ultrasonic image as a key image for a search by using three-dimensional position information of the ultrasonic probe 70 and the ultrasonic image obtained by the position sensor system 30.

According to the embodiments described above, in the image search, both of images as comparison targets are ultrasonic images. However, this is not exhaustive. For example, in the case where one of the comparison targets is an ultrasonic image, the other may be a medical image such as a CT image, an MRI image, etc., which is obtained by a modality other than the ultrasonic diagnostic apparatus. In such a case, for example, the control circuitry 22 according to the first embodiment correlates CT volume data with a three-dimensional model of a subject in advance. The control circuitry 22 defines in advance, information for specifying an imaging position where a CT image according to CT volume data is imaged. Thereafter, the control circuitry 22 performs image recognition of a body mark and a probe mark set in an ultrasonic image as a key image for a search.

Accordingly, information for specifying an imaging position of the ultrasonic image as a key image for a search is determined in a three-dimensional model of a subject so that a tomographic image of a corresponding position can be reconstructed from CT volume data and displayed.

Examples of a method for correlating CT volume data with a three-dimensional model of a subject include a method in which a surface shape of a subject is recognized from volume data and is applied to a portion having a similar surface shape in the three-dimensional model.

Another example is a method in which a scan image for positioning an imaging range is imaged over a range broad enough to make a correlation with a three-dimensional model, and the scan image is correlated with the three-dimensional model in advance by comparing an outline of the scan image with an outline of the three-dimensional model, thereby applying imaged CT volume data to the three-dimensional model.

The description in the above embodiments is premised on use of an ultrasonic diagnostic apparatus. However, this is not exhaustive. For example, an ultrasonic image data piece assigned with at least one of a body mark, a probe mark, and/or an annotation may be transferred to a PC (personal computer), a work station, etc., and the respective functions realized by the control circuits according to the above embodiments may be executed as a medial image processing apparatus.

In the first embodiment, for example, the image recognizing function 221, the sample-model registering function 223, the mapping function 225, and the similarity calculating function 227 realize a calculating unit. In the second embodiment, for example, the image recognizing function 221, the sample-model registering function 223, and the similarity calculating function 227A realize the calculating unit. In the third embodiment, for example, the image recognizing function 221B and the similarity calculating function 227B realize the calculating unit.

The term “processor” used in the above explanation means, for example, circuitry such as a CPU (Central Processing Unit), a GPU (Graphics Processing Unit), an ASIC (Application Specific Integrated Circuit), or a programmable logic device (for example, an SPLD (Simple Programmable Logic Device), a CPLD (Complex Programmable Logic Device), or an FPGA (Field Programmable Gate Array)). A processor realizes its functions by reading and executing the program stored in the storage circuitry. Each processor of the present embodiment is not limited to a configuration as a single circuit; a plurality of independent circuits may be combined into one processor to realize the function of the processor. Furthermore, a plurality of constituent elements shown in FIGS. 1, 10, and 13 may be integrated into one processor to realize their functions.

While certain embodiments have been described, these embodiments have been presented by way of example only, and are not intended to limit the scope of the embodiments. Indeed, the novel embodiments described herein may be embodied in a variety of other forms; furthermore, various omissions, substitutions and changes in the form of the embodiments described herein may be made without departing from the spirit of the invention. The accompanying claims and their equivalents are intended to cover such forms or modifications as would fall within the scope and spirit of the inventions.

1. An ultrasonic diagnostic apparatus comprising processing circuitry configured to:
obtain an ultrasonic image assigned with incidental information as imaging information, the incidental information including at least one of a body mark and a probe mark, the body mark corresponding to an imaged region at a time when a subject is imaged with an ultrasonic probe, the probe mark defining a position where the ultrasonic probe locates on the body mark;
calculate position specifying information for specifying an imaging position with respect to the ultrasonic image by performing image recognition of the incidental information assigned to the ultrasonic image;
extract from a plurality of first images obtained in a past, a second image assigned with incidental information including position specifying information corresponding to the calculated position specifying information; and
display the second image.

2. The ultrasonic diagnostic apparatus according to claim 1, wherein the processing circuitry is configured to correlate at least one of the body mark and the probe mark with a three-dimensional model based on the calculated position specifying information.

3. The ultrasonic diagnostic apparatus according to claim 1, wherein the incidental information further includes an annotation.

4. The ultrasonic diagnostic apparatus according to claim 1, wherein:
the second image is one of a plurality of second images extracted by the processing circuitry; and
the processing circuitry is configured to extract at least one third image that is similar to the ultrasonic image from the plurality of extracted second images, based on image feature amounts based on pixel values of the ultrasonic image and the second images.

5. The ultrasonic diagnostic apparatus according to claim 4, wherein the processing circuitry is configured to:
extract the plurality of second images that are similar to the image specifying information calculated with respect to the ultrasonic image from the first images, based on the position specifying information calculated with respect to the ultrasonic image and the position specifying information included in the first images; and
extract from the plurality of extracted second images, at least one third image that is similar to the ultrasonic image, based on image feature amounts based on pixel values of the ultrasonic image and the second images.

6. The ultrasonic diagnostic apparatus according to claim 1, wherein:
the incidental information includes a body mark assigned with a probe mark; and
the processing circuitry is configured to
perform image recognition of the body mark, and
calculate imaging information regarding an ultrasonic probe from a probe mark included in the body mark.

7. The ultrasonic diagnostic apparatus according to claim 4, wherein the processing circuitry is configured to change how to calculate the imaging information in accordance with a shape of the probe mark.

8. The ultrasonic diagnostic apparatus according to claim 1, wherein:
incidental information assigned to the ultrasonic image includes a direction of an image according to the ultrasonic image and a body mark, and incidental information assigned to the first images includes an direction of the first images and a body mark; and
the processing circuitry is configured to
change a direction of the image according to the ultrasonic image or a direction of the image according to the first images in a manner so that the direction of the image according to the ultrasonic image and the direction of the image according to the first images match with each other and
in accordance with this change, change a shape of a body mark assigned to the ultrasonic image or the first images.

9. The ultrasonic diagnostic apparatus according to claim 3, wherein the processing circuitry is configured to compare image feature amounts based on pixel values in locations other than a lesion region which is prone to change in shape, with respect to the ultrasonic image and the second images.

10. The ultrasonic diagnostic apparatus according to claim 1, wherein the processing circuitry is configured to extract the second image from the plurality of first images using a probe mark selected on a human body model, as a search key.

11. The ultrasonic diagnostic apparatus according to claim 1, wherein the processing circuitry is configured to extract the second images from the plurality of first images using a probe mark selected on a body mark, as a search key.

12. The ultrasonic diagnostic apparatus according to claim 1, wherein:
the incidental information includes an annotation; and
the processing circuitry is configured to extract the second image from the plurality of first images using as a search key, a character string included in the annotation and a position where the annotation locates on the ultrasonic image.

13. The ultrasonic diagnostic apparatus according to claim 1, wherein the position specifying information has a predetermined range.

14. An ultrasonic diagnostic apparatus comprising processing circuitry configured to:
obtain an ultrasonic image assigned with incidental information as imaging information, the incidental information including at least one of a body mark and a probe mark, the body mark corresponding to an imaged region at a time when a subject is imaged with an ultrasonic probe, the probe mark defining a position where the ultrasonic probe locates on the body mark;
calculate image specifying information for specifying an imaging position with respect to the ultrasonic image by performing image recognition of the incidental information assigned to the ultrasonic image; and
store the position specifying information in association with the ultrasonic image in a predetermined storage device.

15. A medical image processing apparatus comprising processing circuitry configured to:
obtain an ultrasonic image assigned with incidental information as imaging information, the incidental information including at least one of a body mark and a probe mark, the body mark corresponding to an imaged region at a time when a subject is imaged with an ultrasonic probe, the probe mark defining a position where the ultrasonic probe locates on the body mark;
calculate position specifying information for specifying an imaging position with respect to the ultrasonic image by performing image recognition of the incidental information assigned to the ultrasonic image;
extract from a plurality of first images obtained in a past, a second image assigned with incidental information including position specifying information corresponding to the calculated position specifying information; and
display the second image.
